(19) United States
(12) Patent Application Publication
(10) Pub. No.: US 2025/0120585 A1
(43) Pub. Date: Apr. 17, 2025

(54) SPACER FOR DENTAL ACQUISITION

(71) Applicant: DENTAL MONITORING, PARIS (FR)

(72) Inventors: Thomas PELLISSARD, Maisons-Alfort (FR); Cédric LANCON, VILLERS FARLAY (FR); Louis-Charles ROISIN, PARIS (FR); Xavier DOREMUS, MONTROUGE (FR)

(21) Appl. No.: 18/719,146

(22) PCT Filed: Dec. 12, 2022
(86) PCT No.: PCT/EP2022/085370

§ 371 Date: Jul. 2, 2024

(30) Foreign Application Priority Data
Dec. 15, 2021 (FR) FR2113577

(51) Int. Cl. A61B 1/24; A61B 1/32

(52) U.S. Cl. CPC A61B 1/24; A61B 1/32

(57) ABSTRACT

A one-piece spacer for acquisition, with a mobile phone, of 2D or 3D representations of teeth of a user, the spacer comprising a tubular part having a longitudinal axis, the tubular part—having a length greater than 3 cm,—opening out via an acquisition opening and via an oral opening, being designed to be fixed detachably to the mobile phone in an acquisition position in which an optical objective of the mobile phone has an at least partial view of the oral opening through the acquisition opening, and—comprising a non-deformable portion, which opens out via the acquisition opening, and, in the continuation of the non-deformable portion, a deformable portion, which opens out via the oral opening and is deformable between a rest configuration in which the oral opening has a maximum area and a contracted configuration in which the oral opening has a minimum area.

### TECHNICAL FIELD

The present invention relates to a kit for acquiring representations of teeth, and in particular dental photos, in particular for implementing a method as disclosed in international application PCT/EP2015/074896. The invention also relates to a spacer for such an acquisition, in particular suitable for such a kit.

### STATE OF THE ART

PCT/EP2015/074897 discloses an acquisition kit enabling a user to acquire, by means of a mobile phone, extraoral photos of their teeth, with the mouth closed or the mouth open, in different orientations.

Acquisition kits are also disclosed in FR3087644A1, EP3391810A1 and US20180303331A1.

There is an ongoing need for an acquisition kit which is
compact,
inexpensive to manufacture,
preferably reusable,
able to be easily handled and brought into the service position in the user's mouth,
then, using a mobile phone, which easy enables extraoral acquisition of 2D or 3D representations by users themselves, in occlusal or lateral views, mouth open or mouth closed, under optimum hygiene conditions.

2D or 3D representations of teeth can be, in particular, extraoral images or three-dimensional digital models.

Finally, the kit must enable data to be acquired for each of the user's teeth, and in particular for teeth at the back of the mouth.

However, the dimensions of users' mouths can vary considerably, based on their morphology and in particular their age. If users do not use an acquisition kit adapted to the dimensions of their mouth, acquisition is not comfortable and photos may be of poor quality.

There is therefore a need for an acquisition kit that enables users to acquire 2D representations, such as photos, or 3D representations, such as three-dimensional models, using a mobile phone, in occlusal or lateral views, with the mouth open or closed, and that limits the risk of acquisition with an unsuitable kit.

One aim of the present invention is to meet this need, at least partially.

### SUMMARY OF THE INVENTION

The invention proposes a spacer, preferably in one piece, for acquiring 2D or 3D representations of teeth of a user with a mobile phone, the spacer comprising a tubular part having a longitudinal axis X,
having a length greater than 3 cm,
   opening out via an acquisition opening and via an oral opening, and
shaped to be detachably attached, directly or via an adapter, to the mobile phone, in an acquisition position wherein an optical objective of the mobile phone has an at least partial view of the oral opening through the acquisition opening,
the tubular part comprising, preferably consisting of, a non-deformable portion, which opens out via the acquisition opening, and, in the continuation of the non-deformable portion, a deformable portion, preferably elastically deformable, which opens out via the oral opening and is deformable between a rest configuration wherein the oral opening has a maximum area and a contracted configuration wherein the oral opening has a minimum area, the ratio of the minimum area, that is, in the contracted configuration, to the maximum area, that is, in the rest configuration, being less than 0.95, preferably less than 0.9, preferably less than 0.8, preferably less than 0.7, preferably less than 0.6, and/or greater than 0.1, preferably greater than 0.3, preferably greater than 0.4, preferably greater than 0.5.

A reduction in this ratio advantageously broadens the range of user morphologies to which the spacer can be adapted, as well as making it easier to move into the service position.

As will be seen in greater detail later in the description, the spacer not only serves to move the mobile phone away from the oral opening, owing to the length and rigidity of the non-deformable portion, but can also be adapted to the user's morphology, by deforming the acquisition opening. Its one-piece design not only considerably reduces manufacturing costs, but also simplifies acquisition by the user.

Preferably in the continuation of the deformable portion, the spacer preferably comprises at least two supports designed to elastically curl up part of a user's lips in a service position wherein the oral opening faces the teeth of the user, so as to expose the teeth that are visible through the acquisition opening and the oral opening. Preferably, the spacer comprises two so-called lateral supports to space the right and left corners of the mouth apart, and preferably two so-called central supports to space the upper and lower lips apart.

The oral opening can thus be modified elastically, depending on the action exerted on the support(s), enabling the spacer to adapt elastically to the dimensions of the user's mouth. This makes the spacer suitable for users with different mouth sizes, such as an adult and a child. As a result, this eliminates the need for each user to choose the right spacer. This advantageously reduces the risk of error during acquisition.

Additionally, the mobility of the supports facilitates introduction of the spacer into the user's mouth, by contracting the oral opening during said introduction, preferably by compressing the deformable portion with a single hand, preferably by pinching between two fingers.

In a preferred embodiment, the spacer performs not only the function of a conventional spacer, but also that of a “retractor”, owing to the elastic mobility of the supports relative to the non-deformable portion.

Finally, the flexibility of the deformable portion means that supports, and in particular lateral supports, can be introduced into the mouth that are much larger than those of the prior art, and in particular lateral supports that are long enough to spread the cheeks away from the teeth, including the molars. This considerably improves the quality of the acquired representations, as shown in FIG. 4.

A spacer according to the invention may comprise one or more of the following optional features: the deformable portion comprises a plurality of tabs, each tab
partially delimiting the oral opening,
being physically and exclusively connected to the non-deformable portion by a junction at one end of said tab, and
being elastically deformable or bendable under the effect of a thrust directed toward said longitudinal axis between the rest and contracted configurations;
the spacer comprises a stop member arranged to block movement of the tabs toward the longitudinal axis when said tabs reach the contracted configuration;
the stop member is arranged in such a way that, in said contracted configuration, each tab is spaced by a distance greater than 1 mm, preferably greater than 2 mm, preferably greater than 3 mm, preferably greater than 5 mm, preferably greater than 8 mm, preferably greater than 10 mm from each of the adjacent tabs, which advantageously limits the risks of pinching of the lips;
the stop member is arranged so that, in said contracted configuration, each tab is spaced by a distance of less than 30 mm, preferably less than 20 mm, from each of the adjacent tabs, which advantageously limits the risks of unsatisfactory spacing of the lips or cheeks between the tabs;
the spacer comprises a member for coupling the movement of at least one tab with the movement of at least one adjacent tab, from the rest configuration to the compacted configuration, and in particular, in the preferred embodiment, so that the movement of the lateral tabs drives the movement of the central tabs, which advantageously allows coordination of the movements of the various tabs;
the spacer comprises a masking flap configured to completely mask the space extending between two adjacent tabs, in particular between any central tab and at least one, preferably each of the lateral tabs adjacent to the central tab, said flap preferably constituting said coupling member, the masking preferably being permanent from the rest configuration to the contracted configuration;
at least one tab, preferably each tab, comprises a support shaped so as, in a service position, to spread part of the user's lips away from the user's teeth;
at least one said support is shaped so as to allow, in the service position, sliding of the user's lips away from the user's teeth, preferably over a distance (L_{50d} in FIG. 6) greater than 3 mm, preferably greater than 5 mm, preferably greater than 10 mm, and/or less than 100 mm;
the tubular part is made of a transparent material;
the deformable portion preferably consists of
said right and left lateral tabs comprising right and left lateral supports shaped so as, in a service position, to spread the right and left corners of the user's lips from the user's teeth, respectively,
said upper and lower central tabs comprising upper and lower supports shaped so as, in the service position, to spread the upper and lower central parts of the user's lips away from the user's teeth, respectively, thus keeping the teeth clear for the optical objective;
a central tab comprises said stop member, said stop member being arranged such that a lateral tab, preferably a masking flap integrated into a lateral tab, comes into abutment with said stop member when said lateral tab reaches the contracted configuration;
a lateral tab comprises said coupling member, preferably a masking flap, said coupling member bearing on at least one central tab, preferably for more than 50%, more than 70%, more than 90%, preferably substantially 100% (that is, permanently bearing) of the travel of said lateral tab between the rest and contracted configurations;
at least one, preferably each lateral support has a free end which, in the rest configuration, extends at a distance (Dy₃₀ in FIG. 6) from the longitudinal axis X greater than 45 mm, preferably greater than 50 mm, preferably greater than 55 mm, preferably greater than 60 mm, preferably greater than 65 mm, and/or less than 80 mm, preferably less than 75 mm, preferably less than 70 mm, so as to elastically spread a user's cheek in the service position; the deformability of the deformable portion advantageously allows a great separation of the cheeks, while allowing easy insertion into the mouth;
the distance (Dy₃₀′ in FIG. 6) between said free end and the longitudinal axis X is preferably less than 60 mm, preferably less than 50 mm, preferably less than 45 mm, in the contracted position;
each lateral support moves progressively away from the longitudinal axis X from the oral opening to a free end of said lateral support, which advantageously limits the risk of trauma;
the spacer comprises a mechanism, which can be selectively activated or deactivated, for holding the deformable portion in the contracted configuration, preferably in the form of an elastic clip or claw;
the mechanism can preferably be activated and/or deactivated by moving the tabs, preferably by moving two opposing tabs toward one another, preferably by moving the right and left lateral tabs toward one another and/or, even more preferably, by moving the top and bottom center tabs toward one another.

The invention also relates to an acquisition kit comprising:
a mobile phone equipped with an optical objective for acquiring a 2D and/or 3D representation;
a spacer according to the invention to which the mobile phone is attached, preferably detachably, optionally via an adapter, the mobile phone being attached to the spacer in such a way that the objective has at least a partial view of the oral opening through the acquisition opening.

A kit according to the invention may comprise one or more of the following optional features:
the kit comprises an adapter and the non-deformable portion of the spacer has a shape complementary to a housing of the adapter, so that it can be inserted therein, said insertion preferably activating said fastener, preferably by clipping;
the objective is less than 2 cm, preferably less than 1 cm, from the acquisition opening.

The invention also relates to a method of acquiring a 2D or 3D representation by means of an acquisition kit according to the invention, said method comprising the following steps:
a) partial insertion, by the user, of the spacer into the mouth of the user to a service position wherein the optical objective of the mobile phone has a view of the teeth of the user through the oral opening, the spacer supports separating the lips of the user to expose said teeth;
b) activation, preferably by the user, of the mobile phone so as to acquire said 2D or 3D representation;
c) optionally, modification of the positioning of the oral opening relative to the user, for example by rotation around the user, then return to step b);
d) optionally, disassembly of the acquisition kit, preferably by the user;
e) optionally, washing and/or disinfection of the spacer, or even only of the spacer.

A method according to the invention may comprise one or more of the following optional features:
preferably, at least one said representation facing the user and at least one said representation to the right or left of the user and/or preferably at least one said open-mouth representation and at least one said closed-mouth representation are acquired;
the tubular part of the spacer is configured to be entirely on the outside of the teeth in the service position, so as to allow full occlusion;
in the service position, the teeth do not rest on the spacer, so that the user can, by turning the head relative to the spacer, modify the teeth that are visible through the oral opening and/or modify the spacing between his dental arches;
in the service position, the spacer does not press on the teeth so as to move the two jaws apart;
in the service position, the optical objective of the mobile phone has a view of more than 2, more than 3, more than 5, more than 10, more than 15, more than 20, preferably more than 30 teeth of the user.

### DEFINITIONS

The “service position” is the position wherein the oral opening extends into the mouth of the user, as shown in FIG. 5.

An “assembled position” is a position wherein the mobile phone is rigidly attached to the spacer or to an adapter attached to the spacer.

An “acquisition position” is an assembled position wherein the mobile phone objective has at least a partial, preferably complete, view of the oral opening, through the acquisition opening and the oral opening.

The “service configuration” is the configuration of the spacer in the service position.

The “rest configuration” is the configuration of the spacer when it is under no stress other than gravity and the corresponding reaction. In the figures, the spacer is shown in the rest configuration. Unless otherwise indicated, all spacer dimensions are measured in the rest configuration.

The “contracted configuration” is the configuration of the spacer when it undergoes a set of stresses conferring a minimum area to the oral opening, without breaking the spacer.

A “portion” is a fraction of the tubular part delimited by two planes perpendicular to the axis X. A portion is “deformable” or “non-deformable” depending on whether or not it deforms when a user compresses it between his fingers, without breaking it. Deformation is a change in the shape of the portion that modifies the internal volume of said portion, and in particular reduces it. Deformation of the deformable portion may, for example, lead to a reduction of more than 1%, more than 2%, more than 5%, preferably more than 10%, preferably more than 20% and/or less than 40%, preferably less than 30% of the internal volume of this portion. Deformation is preferably elastic.

The interface between the non-deformable portion and the deformable portion is a plane perpendicular to the axis X positioned, along the axis X, at the point where the tubular part begins to deform, in particular at the junction of the tab closest to the acquisition opening.

An object is “elastically mobile” if its position relative to a reference, e.g. another object, can be modified by the action of a force and if, when this force ceases, it returns to its initial position. A support elastically curls up a lip insofar as it returns to its initial position relative to the non-deformable portion when the action of the lip on it ceases. In other words, the support is elastically movable relative to the non-deformable portion.

“One-piece” means that the spacer is made in a single piece, with the deformable portion in one piece with the non-deformable portion.

The “mouth closed” position is the occlusion position wherein the user's upper and lower teeth are in contact. An “open mouth” position is one wherein the user's upper and lower teeth are not in contact, preferably the position with the mouth fully open.

A “mobile phone” is a device like the iPhone®. Such a device typically weighs less than 500 g. It is equipped with a camera to take films or photos, or even a scanner to acquire three-dimensional digital models through an optical objective. A mobile phone is further capable of exchanging data with another device more than 500 km away from the mobile phone.

Mobile phones can be used to acquire 2D or 3D representations “extraorally”, that is, without the mobile phone objective being inserted into the user's mouth.

The optical objective of the mobile phone “has a view” of an object” when triggering the acquisition of a 2D or 3D representation leads to the recording of a 2D or 3D representation that depicts this object.

A “2D representation” and a “3D representation” are digital objects in two or three dimensions, respectively, acquired by a mobile phone, extraorally, through the mobile phone's optical objective. The “2D representations” acquired by the mobile phone are images, in particular conventionally photos and films. The “3D representations” acquired by the mobile phone are three-dimensional digital models, or “3D models”.

An “image” refers to a two-dimensional image, such as a photograph. An image is made up of pixels. A “film” is considered to be a collection of photos.

A 3D model is made up of a set of voxels.

An element integral with another is considered to be rigidly “attached” to this other element.

A “user” refers to a person who uses a kit according to the invention to acquire a 2D or 3D representation of at least part of his teeth, whether or not this person is ill, or whether or not this person is undergoing orthodontic treatment.

“Masking a slit” refers to “being shaped to substantially mask the view of the interior of the tubular part when the viewing direction is perpendicular to the outer surface of the tubular part around said slit.

Unless otherwise indicated, qualifiers used to define positions or orientations in space, such as “horizontal”, “vertical”, “bottom”, “top”,

“upper”, “lower”, “right” or “left” are defined for clarity with reference to a service position observed by a user with his head upright and lips around the oral opening of the spacer. For example, a vertical axis is an axis which is vertical in the service position.

When reference is made to a first object, e.g. a tab, and then to a second “adjacent” object, e.g. an adjacent tab, this means that the adjacent object is next to the first object when turned around the axis X. In the preferred embodiment, each lateral tab is adjacent to the two central tabs, and vice versa

Unless otherwise indicated, “outside” and “inside” refer to volumes or closed contours, to designate an element outside the volume, for example outside a chamber, or outside the closed contour. For example, the outer surface of the tubular part is the surface of the tubular part that is exposed to the outside.

The adjectives “first” and “second” are used for clarity only. In particular, the presence of a first represented brand does not imply the presence of other represented brands.

For the sake of clarity, the orthonormal coordinate system Oxyz) is a coordinate system such that, in the service position, the axis Ox) extends horizontally and perpendicularly to the general plane of the oral opening, the axis Oz) extends vertically, and the axis Oz) extends perpendicularly to the axes Ox) and Oz). For the user, the axes Ox), Oy) and Oz) are therefore front-to-back, right-to-left, and vertical, respectively.

The axis of an opening is the axis that passes through its center, perpendicular to the general plane of the opening.

First and second straight lines are said to be perpendicular to one another when first and second planes perpendicular to said first and second straight lines, respectively, are perpendicular to one another.

“Comprise”, “include” and “have” are to be interpreted broadly and without limitation, unless otherwise specified.

“Attaching” one part to another means establishing a physical connection between the two, so that any movement of one part will eventually cause the other to move. A part mounted in translation, in rotation or on a swivel on another part is “attached” to this other part. An attachment can be “permanent” or “releasable”, depending on whether or not the two parts are suitable for disassembly, preferably by hand, by a user. A fastener can be rigid or non-rigid, depending on whether or not it allows relative movement of the two parts which are attached to one another. An element that is integral with another is considered to be “attached” on this other element.

### BRIEF DESCRIPTION OF THE FIGURES

Further features and advantages of the invention will become apparent from the following detailed description and from an examination of the appended drawing, wherein:

FIG. 1 shows a kit according to the invention, in perspective, in the assembled position, from the back left from above (1A), from the back right from below (1B), from the bottom right from the front (1C), and from the front from below (1D);

FIG. 2 shows a perspective view of the spacer from the kit shown in FIG. 1;

FIG. 3 shows a perspective view of the adapter from the kit shown in FIG. 1;

FIG. 4 shows tests with a conventional spacer (left photos) and with a spacer according to the invention (right photos);

FIG. 5 schematically shows the service position, with the user viewed from the front (FIG. 5A) and from the side (FIG. 5B);

FIG. 6 shows the spacer of FIG. 1, seen from above, in the rest configuration, with the dashed lines showing the flexibility of the lateral tabs and the measurements, in mm, corresponding to a preferred flexibility;

FIG. 7 shows the spacer of FIG. 1, seen from the side, in the rest configuration, with the dashed lines showing the flexibility of the central tabs and the measurements, in mm, corresponding to a preferred flexibility;

FIG. 8 shows the spacer of FIG. 1, seen from the front, in its rest configuration;

FIG. 9 shows a side view of the spacer of FIG. 1 in retracted configuration;

FIG. 10 shows the deformability of a central tab of the spacer shown in FIG. 1;

FIG. 11 shows an embodiment wherein the locked position of a tab can be locked by means of a claw.

In the various figures, identical or similar components have been designated with identical references.

For the sake of clarity, a generic reference may be used in the description to designate a member in a generic way, for example a tab, with only specific references, corresponding to variations of the generic reference (for example to designate upper, lower, right and left tabs), being shown in the figures.

The figures are provided for illustrative purposes. They are not exhaustive.

### DETAILED DESCRIPTION

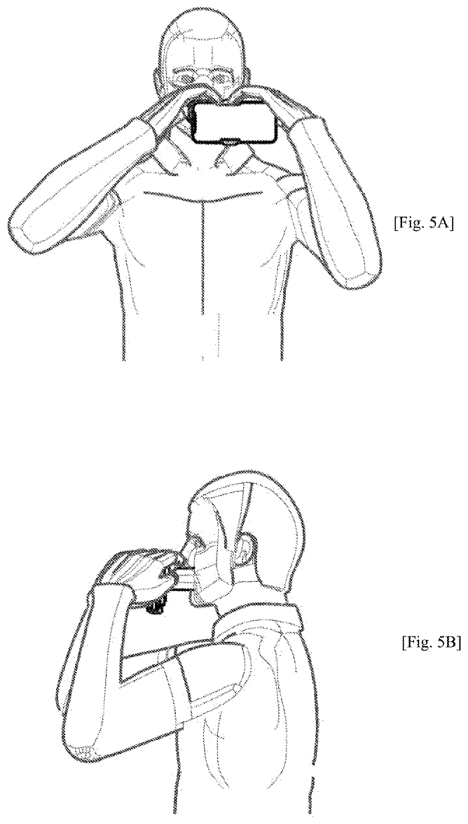

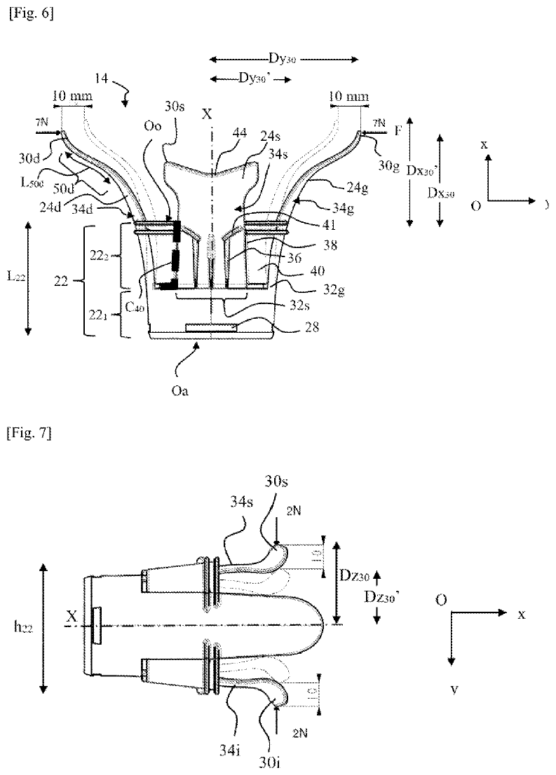

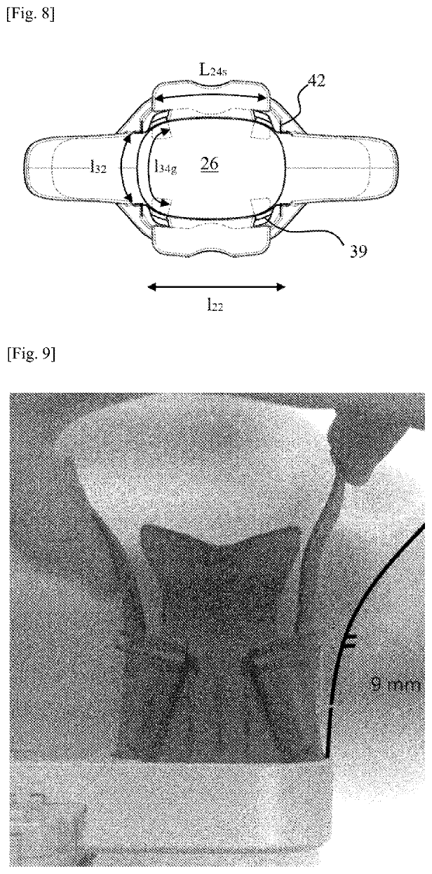

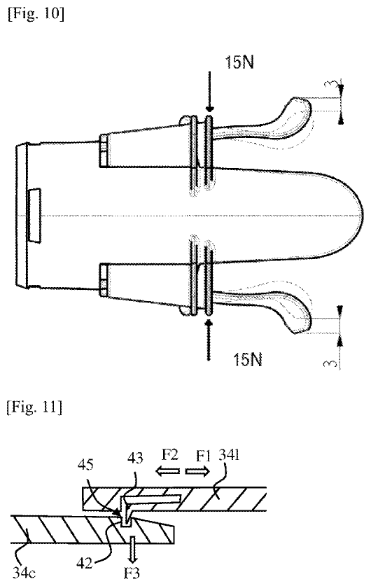

Generally speaking, a spacer according to the invention may in particular comprise one or more of the following optional features:
in the rest configuration, the area of the oral opening is preferably greater than 1500 mm², preferably greater than 1800 mm², preferably greater than 2000 mm², preferably greater than 2100 mm², preferably greater than 2200 mm², and/or less than 3000 mm², preferably less than 2800 mm², preferably less than 2600 mm², preferably less than 2500 mm², preferably less than 2400 mm²;
in the contracted configuration, the area of the oral opening is preferably greater than 500 mm², preferably greater than 800 mm², preferably greater than 1000 mm², preferably greater than 1100 mm², preferably greater than 1200 mm², preferably greater than 1300 mm², and/or less than 2000 mm², preferably less than 1800 mm², preferably less than 1600 mm², preferably less than 1500 mm², preferably less than 1400 mm²;
the length of the tubular part, measured along the axis X, between the acquisition opening and the oral opening is greater than 3 cm, preferably greater than 4 cm, preferably greater than 5 cm, and/or less than 20 cm, preferably less than 15 cm, preferably less than 10 cm, preferably less than 8 cm, preferably less than 7 cm, preferably less than 6 cm, preferably less than 5.5 cm;
the length of the non-deformable portion, measured along axis X, between the acquisition opening and the deformable portion is greater than 0.5 cm, preferably greater than 1 cm, preferably greater than 1.5 cm, preferably greater than 2 cm, and/or less than 20 cm, preferably less than 10 cm, preferably less than 5 cm, preferably less than 4 cm, preferably less than 3 cm, preferably less than 2.5 cm;
the length of the deformable portion, measured along the axis X, up to the oral opening is greater than 0.5 cm, preferably greater than 1 cm, preferably greater than 1.5 cm, preferably greater than 2 cm, preferably greater than 2.5 cm, and/or less than 20 cm, preferably less than 10 cm, preferably less than 5 cm, preferably less than 4 cm, preferably less than 3.5 cm;
the spacer is symmetrical with respect to a vertical plane;
the spacer is symmetrical with respect to a horizontal plane;
in the rest configuration, the oral opening has an area greater than 7 cm², preferably greater than 10 cm², preferably greater than 15 cm², preferably greater than 20 cm² and/or less than 30 cm²;
in one embodiment, the tubular part comprises a wall which delimits a substantially closed chamber, that is, substantially devoid of openings apart from the oral and acquisition openings;
the chamber is preferably empty, and in particular does not enclose any lens or lighting device;
the chamber has the shape of a cylindrical or frustoconical tube;
the inner and/or outer surface of the wall delimiting said chamber is smooth to the touch and preferably free of any asperities or cavities;
the spacer is configured so as to leave the view through the oral opening completely clear from the acquisition opening, preferably without a tongue support;
the spacer, in particular the non-deformable portion, comprises a fastener by means of which
the mobile phone is attached to the spacer or
an adapter, to which the mobile phone is attached, is attached to the spacer;
the non-deformable portion and/or the deformable portion have a wall thickness greater than 0.3 mm, preferably greater than 0.5 mm, preferably greater than 0.8 mm and/or less than 8 mm, preferably less than 5 mm, preferably less than 3 mm, preferably less than 2 mm, preferably less than 1.5 mm, preferably constant;
the deformable portion comprises slits extending from the oral opening, preferably to the non-deformable portion, so as to define flexible tabs;
one slit, preferably each slit, is straight, L-shaped or T-shaped;
one slit, preferably each slit, extends partially or totally in a plane passing through the axis X;
a slit delimiting a tab extends partially in a plane passing through the axis X, then extends, preferably in a plane perpendicular to the axis X, so that the junction between said tab and the non-deformable portion has a length less, preferably at least 1.1 times less, preferably at least 1.2 times less, than the width of the tab at mid-length of the tab;
one slit, preferably each slit, widens toward the oral opening;
one slit, preferably each slit, has a maximum width (measured perpendicular to the direction of the slit length) and/or a minimum width of less than 3 mm, preferably less than 2 mm, preferably less than 1 mm, and/or greater than 0.1 mm;
none of the tabs protrude into the chamber defined by the tubular part;
each tab partially defines said chamber;
one tab, preferably each tab, is physically connected to the non-deformable portion by a junction extending over a width (I₃₂ in FIG. 8) greater than 5 mm, preferably greater than 10 mm, preferably greater than 15 mm, preferably greater than 20 mm, preferably greater than 25 mm, and/or less than 80 mm, preferably less than 60 mm, preferably less than 50 mm, preferably less than 40 mm;
the junction of a tab, preferably of each tab, extends in a transverse plane, that is, perpendicular to the axis X;
the inner surface of a tab, preferably each tab, extends in the continuation of the inner surface of the non-deformable portion, so that the junction of the tab does not create any asperities or cavities;
the number of tabs is greater than 2 and/or less than 10, preferably less than 5, preferably is 4;
the deformable portion comprises 4 tabs, equiangularly distributed around the axis X, each tab preferably being symmetrical to the tab facing it;
the deformable portion comprises two lateral tabs defining the right and left ends of the oral opening, and two central tabs defining the upper and lower ends of the oral opening;
the lengths of the various tabs, from their junctions to the oral opening, are preferably identical, but may be different;
preferably, at least the lengths of the different lateral tabs are identical, and preferably, at least the lengths of the different central tabs are identical;
at least one, preferably each, central tab carries one or more stiffening ribs, preferably extending in a plane passing through the axis X;
the deformability of one central tab, preferably of each central tab, is such that the free end of the central support of a central tab is displaced by 3 mm, under the effect of a centripetal force, that is, a force directed toward the axis X, exerted in the general plane of the oral opening, greater than 5 N, preferably greater than 10 N, preferably greater than 13 N, and/or less than 30 N, preferably less than 25 N, preferably less than 20 N, preferably less than 17 N, the distance being measured perpendicular to the axis X, as shown in FIG. 10;
the deformability of one central tab, preferably of each central tab, is such that the free end of the central support of a central tab moves by 10 mm, under the effect of a centripetal force, that is, a force directed toward the axis X, exerted at the end of the central tab, greater than 0.5 N, preferably greater than 1.0 N, preferably greater than 1.5 N, and/or less than 5 N, preferably less than 3 N, preferably less than 2.5 N, the distance being measured perpendicular to the axis X, as shown in FIG. 7;
at least one tab, preferably at least one lateral tab, preferably each lateral tab, or even each tab comprises at least one masking flap masking at least partially, preferably totally, a slit delimiting said tab, preferably at least in the service position, preferably irrespective of the deformation of the deformable portion;
the masking flap of a tab, preferably each masking flap, extends to partially cover an adjacent tab, preferably at least in the service configuration, preferably regardless of the deformation of the deformable portion;
a tab comprises a coupling member, for example a pin, preferably integrated into a masking flap, configured to bear on an adjacent tab when the deformable portion is deformed from the rest configuration to the contracted configuration, preferably at least in the rest configuration, preferably irrespective of the configuration of the deformable portion between the rest configuration and the service configuration;
the coupling member of a tab, preferably each coupling member resting on an adjacent tab, is shaped so that a centripetal thrust on said tab pushes and moves the adjacent tab toward the axis X;
one tab, preferably each tab resting on an adjacent tab, is shaped so that, during said displacement, said tab, and preferably a masking flap of said tab, slides on the adjacent tab, said adjacent tab comprising a stop member limiting the travel of said tab;
a central tab comprises a so-called “central” stop member, preferably limiting the approach of an adjacent lateral tab toward the axis X, preferably by stopping said lateral tab, preferably by stopping a masking flap of said lateral tab, on said stop member;
the spacer is in the contracted position when the stop member limits the approach of an adjacent lateral tab toward the axis X, a gap being maintained, in said position, between the lateral tab and the adjacent central tab, the gap preferably being greater than 1 mm, 3 mm, 5 mm, so as to limit the risk of pinching of the tongue;
one lateral tab, preferably each lateral tab, comprises so-called “upper” and “lower” coupling members, preferably integrated into masking flaps, shaped to press on an upper central tab and a lower central tab, respectively, so as to push said upper and lower central tabs toward the axis X when said central tab is pushed closer to the axis X; the reaction exerted by a central tab on a lateral tab limits the possibility of flexing the lateral tab, so that the flexibility of the lateral tabs is less than that of the central tabs;
one tab, preferably each tab, comprises, beyond the oral opening, a support designed to rest on part of the user's lips in the service position;
at least one lateral tab, preferably each lateral tab, comprises a “lateral” support, the lateral support being shaped to spread the user's cheeks in the service position;
said lateral support, preferably each lateral support, is shaped like a tongue; advantageously, the risk of the lip escaping during lateral displacement of the spacer is reduced or even eliminated,
said lateral support, preferably each lateral support, is preferably concave toward the axis X;
in the rest configuration, the distance, in the direction of the axis X (axis Ox)), between the oral opening and the free end of said lateral support, preferably of each lateral support, is greater than 20 mm, preferably greater than 25 mm, preferably greater than 30 mm, preferably greater than 35 mm, and/or less than 60 mm, preferably less than 50 mm, preferably less than 55 mm;
in the rest configuration, the distance, measured perpendicularly to the direction of the axis X (axis Oy)), between the axis X and the free end of said lateral support, preferably of each lateral support, is greater than 40 mm, preferably greater than 50 mm, preferably greater than 60 mm, and/or less than 90 mm, preferably less than 80 mm, preferably less than 70 mm; advantageously, the deformability of the deformable portion enables the spacer to comprise lateral supports whose free end deviates significantly from the axis X, the spacer being able to be inserted into the mouth owing to said deformability;
in the contracted configuration, the distance (Dx₃₀′ in FIG. 6), in the direction of the axis X (axis Oy)), between the general plane of the oral opening and the free end of said lateral support, preferably of each lateral support, is greater than 30 mm, preferably greater than 35 mm, preferably greater than 40 mm, preferably greater than 45 mm, and/or less than 70 mm, preferably less than 60 mm, preferably less than 50 mm;
in the contracted configuration, the distance, measured perpendicularly to the direction of the axis X (axis Oy)), between the axis X and the free end of said lateral support, preferably of each lateral support, is greater than 30 mm, preferably greater than 35 mm, preferably greater than 40 mm, and/or less than 70 mm, preferably less than 60 mm, preferably less than 50 mm;
the deformability of a lateral tab, preferably of each lateral tab, is such that the free end of the lateral support of a lateral tab moves by 10 mm, under the effect of a centripetal force, that is, a force directed toward the axis X, exerted at the free end of the lateral tab (shown by arrow F in FIG. 6), greater than 2 N, preferably greater than 3 N, preferably greater than 4 N, preferably greater than 5 N, preferably greater than 5.5 N, and/or less than 12 N, preferably less than 10 N, preferably less than 9 N, preferably less than 8 N, the distance being measured perpendicular to the axis X, as shown in FIG. 6;
one lateral support, preferably each lateral support, has an outer surface and/or an inner surface (facing the axis X) which are smooth to the touch;
the outer surface of one lateral support, preferably of each lateral support, does not present any obstacle projecting from said surface, preferably at least in the sliding zone (L_{50d} in FIG. 6) over which the corner of the mouth resting on said lateral support slides when, in the service position, the user rotates the kit around himself; in particular, the lateral support does not form a channel likely to hook said corner of the mouth, like conventional dental retractors;
the outer surface of one lateral support, preferably of each lateral support, extends away from the axis X, from the oral opening, preferably from the junction of the lateral support with the non-deformable portion, to its free end;
in the rest configuration, the gradient of said separation (dy/dx) along the axis X increases from the oral opening, preferably from the junction of said lateral support, preferably of each lateral support, with the non-deformable portion, to a position along the axis X within 2 cm of the position along the axis X of said free end of the lateral support in question;
in the rest configuration, the gradient of said separation (dy/dx) along the axis X decreases from a position, along the axis X, within 1 cm of the position, along the axis X, of said free end of the lateral support under consideration to said position of said free end;
the greatest width of a lateral support, preferably of each lateral support, is greater than 10 mm, preferably greater than 15 mm, preferably greater than 20 mm, and/or less than 40 mm, preferably less than 30 mm, preferably less than 25 mm;
the greatest thickness of the lateral support, preferably of each lateral support, is greater than 0.5 mm, preferably greater than 1 mm, and/or less than 4 mm, preferably less than 3 mm, preferably less than 2 mm;
at least one central tab, preferably each central tab, carries a “central” support, the central support being shaped to spread a lip of the user in the service position;
said central support, preferably each central support, has a general “T” shape (for example as shown in FIG. 6), preferably concave toward the axis X;
in the rest configuration, the distance, in the direction of the axis X (axis Ox)), between the oral opening and the free end of said central support, preferably of each central support, is greater than 15 mm, preferably greater than 20 mm, preferably greater than 25 mm, and/or less than 40 mm, preferably less than 35 mm, preferably less than 30 mm;
in the rest configuration, the distance (Dz₃₀ in FIG. 7), measured perpendicularly to the direction of the axis X (axis Oz)), between the axis X and the free end of said central support, preferably of each central support, is greater than 15 mm, preferably greater than 20 mm, preferably greater than 30 mm, and/or less than 50 mm, preferably less than 40 mm, preferably less than 35 mm;
in the contracted configuration, the distance, in the direction of the axis X (axis Ox)), between the oral opening and the free end of said central support, preferably of each central support, is greater than 15 mm, preferably greater than 20 mm, preferably greater than 25 mm, and/or less than 40 mm, preferably less than 35 mm, preferably less than 30 mm;
in the contracted configuration, the distance (Dz₃₀′ in FIG. 7), measured perpendicularly to the direction of the axis X (axis Oz)), between the axis X and the free end of said central support, preferably of each central support, is greater than 10 mm, preferably greater than 15 mm, preferably greater than 20 mm, and/or less than 35 mm, preferably less than 30 mm, preferably less than 25 mm;
said central support, preferably each central support, has an outer surface and an inner surface (facing the axis X) which are smooth to the touch;
the outer surface of said central support, preferably of each central support, does not present any obstacle protruding from said outer surface, preferably at least in the sliding zone over which the lip bearing on the central support in question slides when, in the service position, the user raises or lowers the spacer in order to take occlusal views; in particular, the central support in question does not form a channel likely to hook said lip, like conventional retractors;
the outer surface of said central support, preferably of each central support, extends away from the axis X, from the oral opening, preferably from the junction of the central support with the non-deformable portion, to its free end;
in the rest configuration, the gradient of said separation (dy/dx) along the axis X increases from the oral opening, preferably from the junction of the central support in question, to the position of the free end of said central support in question;
the greatest width of the central support, preferably of any central support, is greater than 20 mm, preferably greater than 30 mm, preferably greater than 40 mm, and/or less than 70 mm, preferably less than 60 mm, preferably less than 50 mm;
the greatest thickness of the central support, preferably of any central support, is greater than 0.5 mm, preferably greater than 1 mm, and/or less than 4 mm, preferably less than 3 mm, preferably less than 2 mm;
the spacer comprises right, left, upper and lower supports intended to rest, in the service position, on the right corner of the mouth, the left corner of the mouth, the central part of the upper lip and the central part of the lower lip of a user, respectively, and integrated into said right, left, upper and lower tabs, respectively;
said tabs are shaped so that moving the right and left supports toward one another causes the top and bottom supports to move toward one another, or vice versa;
the spacer comprises a fastener suitable for attaching, preferably detachably, a mobile phone or an adapter to which a mobile phone is attached, preferably detachably, in an acquisition position wherein an optical objective of the mobile phone has at least a partial view of the oral opening through the acquisition opening.

Preferably, as shown in FIG. 9, the lateral ends of the oral opening move by more than 5 mm, more than 7 mm, more than 8 mm and/or less than 12 mm, preferably less than 11 mm, preferably less than 10 mm, preferably 9 mm, in the direction Oy), between the rest position and the contacted position.

We will now turn to the figures.

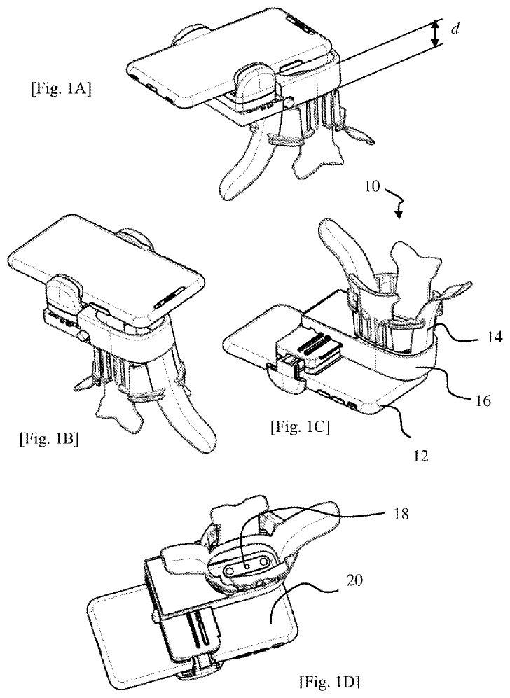

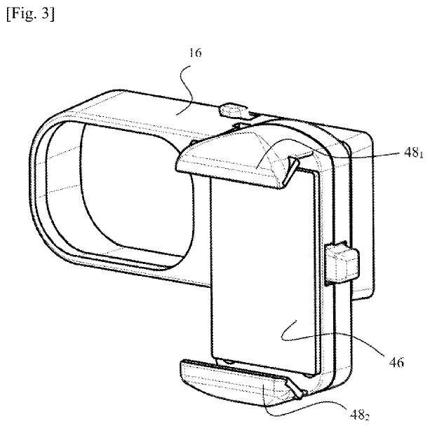

The kit 10 shown in FIG. 1 comprises a mobile phone 12, a spacer 14 and an adapter 16, shown in detail in FIG. 3.

### Mobile Phone

Any conventional mobile phone can be used.

The mobile phone 12 is a personal device, conventionally of a substantially parallelepiped shape, conventionally incorporating a camera.

A mobile phone conventionally has:
a front face fitted with at least one optical objective 18 and, preferably, a flash,
a rear face with a screen 20, and
a side edge, defining the thickness of the phone and joining the front and rear faces.

The screen 20 covers substantially the entire rear face. It acts as an interface for the user. In particular, it enables him to visualize what the optical objective 18 “sees”, that is, the “view” of the optical objective. Preferably, the screen 20 is touch-sensitive and can be used to control mobile phone functions.

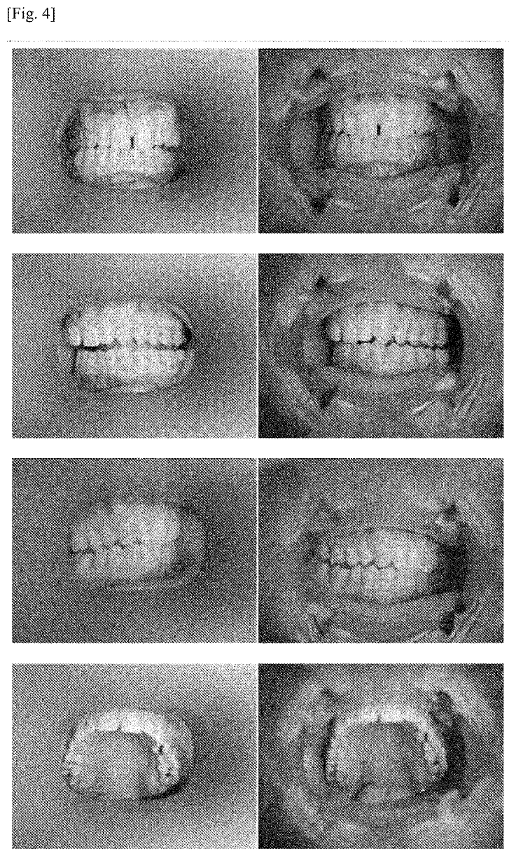

The optical objective 18 typically has an optical axis perpendicular to the front face, and enables the acquisition of 2D representations, as shown in FIG. 4, or even 3D representations. In particular, it enables the acquisition of color and/or infrared photos. Infrared photos are ideal for showing the teeth with excellent contrast.

The mobile phone 12 is attached, preferably detachably, to the spacer 14, preferably via an adapter 16.

### Spacer

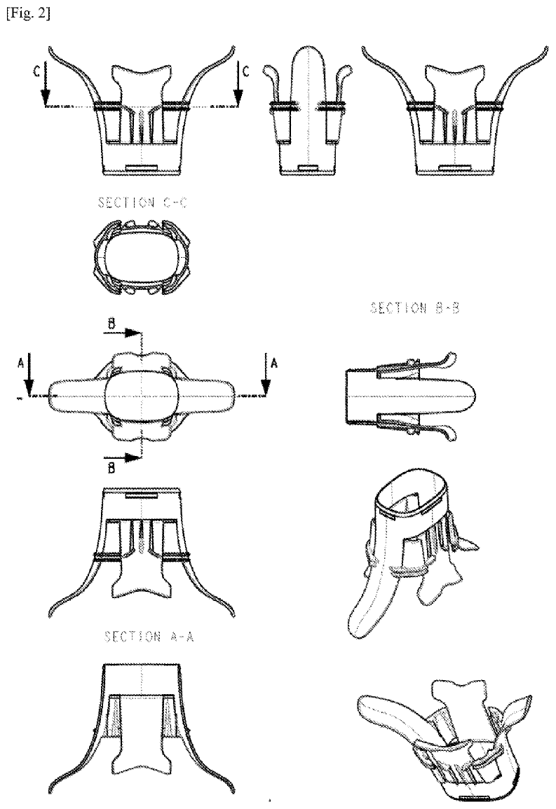

The spacer, shown separately in FIG. 2, simultaneously makes it possible to
spread the user's lips to expose the teeth, and
define, optionally with the adapter, the position of the mobile phone in relation to the oral opening Oo, and therefore in relation to the teeth.

The spacer 14, of longitudinal axis X, comprises a tubular part 22, and a set of supports 24 designed to bear on respective parts of the lips in the service position.

The spacer 14 is a one-piece unit, with all the supports integrally formed with the tubular part. Advantageously, its manufacturing cost is limited and its use is facilitated.

Preferably, the spacer is shaped to be removable, preferably so as to be able to be manufactured by injection of a moldable material, preferably a polymer, into a mold.

### Tubular Part

The tubular part 22 defines a chamber 26. The cross section of the chamber, that is, in a sectional plane perpendicular to the axis X, can be constant or variable along the axis X.

Preferably, in a portion of the chamber extending over more than 70%, more than 80%, more than 90%, more than 95%, more than 99%, preferably 100% of the length of the chamber, the spacer has an inner surface that is smooth to the touch, free of protruding edges and/or free of re-entrant edges, and in particular free of the projections disclosed below, a portion of the chamber being a fraction of the chamber delimited by two planes perpendicular to the axis X.

The tubular part 22, of axis X, opens out through an acquisition opening Oa and, on the opposite side, through the oral opening Oo, preferably coaxial with the acquisition opening. In one embodiment, the chamber 26 only opens outwards through the oral Oo and acquisition Oa openings.

The chamber 26 may, however, comprise lateral openings, such as the slits 38 disclosed below, which place it in fluid communication with the environment outside the chamber, provided that the overall shape of the chamber is tubular. Preferably, the total area of the lateral openings represents less than 10%, less than 5% or even less than 2% of the lateral surface of the chamber between the oral Oo and acquisition Oa openings.

Preferably, the spacer is shaped so that, when observed radially with respect to the axis X, the chamber appears, whatever the observation point, to be delimited by a substantially solid wall, with no lateral openings or only slits with a width of less than 4 mm, preferably less than 3 mm, preferably less than 2 mm, preferably less than 1 mm.

Preferably, the spacer is shaped so that the oral opening Oo is, in the service position, and in particular when the user's lips are resting on the central and/or lateral supports, less than 3 cm, preferably less than 2 cm, preferably less than 1 cm from the general plane of the user's mouth, preferably in or against the user's mouth.

Preferably, as shown in FIG. 1, the spacer is shaped so that, in the assembled position, the optical objective 18 of the mobile phone extends proximate to the acquisition opening Oa. The distance between the optical objective 18 and the acquisition opening Oa, measured parallel to the axis X, may be less than 0.5 cm, preferably less than 0.2 cm, preferably substantially zero, particularly if the mobile phone is attached directly to the spacer, preferably in such a way as to close off the acquisition opening Oa.

Preferably, however, the kit comprises an adapter 16 which, in the assembled position shown in FIG. 1, imposes a distance d between the acquisition opening Oa of the spacer 24 and the optical objective 18, measured parallel to the axis X, greater than 0.5 cm, greater than 1 cm, greater than 2 cm, and/or less than 4 cm, preferably less than 3 cm.

Preferably, the smallest dimension of the oral opening, in the rest configuration, and/or of the acquisition opening, for example the width for a rectangular-shaped opening, is greater than 1 cm, preferably greater than 2 cm, preferably greater than 3 cm, and/or less than 5 cm, preferably less than 4 cm.

Preferably, the largest dimension of the oral opening, in the rest configuration, and/or of the acquisition opening, for example the diagonal for a rectangular-shaped opening, is greater than 2 cm, preferably greater than 3 cm, preferably greater than 4 cm, preferably greater than 5 cm, and/or less than 10 cm, preferably less than 9 cm, preferably less than 8 cm, preferably less than 7 cm, preferably less than 6 cm.

Preferably, the ratio, in the rest configuration, of the oral opening area to the acquisition opening area is greater than 1, preferably greater than 1.2, preferably greater than 1.3 and/or less than 1.7, preferably less than 1.6, preferably less than 1.5.

The function of tubular part 22 is to define a spacing between the mobile phone and the oral opening Oo and, preferably, a predefined orientation of the mobile phone relative to the oral opening. Advantageously, in the service position, the representations (images or 3D models) acquired by the mobile phone 12 through the optical objective 18 are thus acquired at a predetermined distance from the user's teeth and according to a predefined orientation. Preferably, the spacer 14 is configured so that this spacing and orientation are constant.

The tubular part 22 may have a constant or variable cross section, preferably substantially rectilinear with axis X, in particular in the general shape of a cylindrical tube, for example with a circular, oval or polygonal cross section, e.g. rectangular, or a frustoconical tube.

The cross section of the tubular part 22, that is, in a sectional plane perpendicular to the axis X, preferably has a substantially identical general shape throughout the tube, for example rectangular or oval or round.

As shown in FIG. 6, the length L22 of the tubular portion 22 of the spacer 14, measured along the axis X, is preferably greater than 3 cm, preferably greater than 4 cm, preferably greater than 5 cm, and/or less than 25 cm, preferably less than 20 cm, preferably less than 15 cm, preferably less than 10 cm, preferably less than 8 cm, preferably less than 7 cm.

The width I₂₂ of the tubular part 22 of the spacer 14, that is, its largest dimension in a transverse plane, considering all transverse planes along the axis X, is preferably greater than 2 cm and/or less than 10 cm.

The height h₂₂ of the tubular part 22 of the spacer 14, that is, its smallest dimension in a transverse plane, considering all transverse planes along the axis X, is preferably greater than 2 cm, preferably greater than 4 cm, preferably greater than 6 cm, preferably greater than 7 cm and/or less than 10 cm, preferably less than 9 cm.

Preferably, the thickness of the tubular part of the spacer is constant, preferably over the entire length of the tubular part, preferably greater than 0.5 mm, preferably greater than 1 mm and/or less than 8 mm, preferably less than 5 mm.

Preferably the tubular part 22, preferably the entire spacer 14, is made of a transparent material. Advantageously, the oral opening can thus be illuminated by a mobile phone flash that is not facing the acquisition opening.

The tubular part 22, preferably the entire spacer 14, is preferably made of a rigid material, preferably a polymer, for example polyethylene PE, polyethylene terephthalate PET, polystyrene PS or polypropylene PP, preferably polycarbonate PC, advantageously transparent and sterilizable.

In one embodiment, the tubular part 22, preferably the entire spacer 14, is made from a recyclable, preferably biodegradable, material.

The tubular part 22 comprises a non-deformable portion 22₁ and a deformable portion 22₂.

The non-deformable portion has sufficient rigidity to ensure that the non-deformable portion does not deform during normal use of the tubular part, in particular when a telephone or adapter is attached against the acquisition opening, or when the user clamps the non-deformable portion in the service position. In particular, no appreciable deformation is detectable to the naked eye when a user squeezes the non-deformable portion in his hands.

The non-deformable portion comprises a fastener 28 which, in said acquisition position, is able to engage with the mobile phone or, preferably, with an adapter to which the mobile phone is attached, in order to hold the mobile phone in position.

The fastener is preferably configured to retain its shape when used for said fastening, making it more reliable. It is preferably formed by a recess and/or bead of material, preferably on the outer surface of the non-deformable portion. The distance between the fastener and the acquisition opening is preferably less than 3 cm, preferably less than 2 cm, preferably less than 1 cm, preferably less than 0.5 cm.

The deformable portion is a portion of the tubular part, that is, a fraction of the tubular part between two planes perpendicular to the axis X, which the user can deform, by hand and without tools, preferably with a single hand, preferably elastically, to modify its shape.

In the rest configuration of the deformable portion, for example when the spacer is placed on a table, the oral opening has a maximum area. However, by compression, preferably with a single hand, the user can reduce the oral opening area, as explained below, to a “contracted” configuration, wherein the oral opening area is minimal. The deformation is elastic, so that when the user releases the compression, the spacer returns to the rest configuration.

The difference between the maximum area and the minimum area enables the spacer to be fitted with large supports, in particular to spread the cheeks, while allowing easy insertion of said supports into the mouth.

### Supports

The deformable portion carries supports designed to be inserted between the user's lips and teeth. Advantageously, the lips can thus be spread so as not to obstruct the oral opening Oa. Additionally, the supports facilitate retention of the spacer 14 in the user's mouth.

The supports are integral with the tubular part 22 of the spacer 14, which limits manufacturing costs and reduces the number of edges. Hygiene is improved as a result. They can be made of an elastomer for optimum comfort.

Preferably, the set of supports comprises two lateral supports, namely a right support 24d and a left support 24g, designed to rest on the right and left corners of the user's lips, respectively, and two central supports, namely an upper support 24s and a lower support 24i, designed to rest on the central parts of the user's upper and lower lips.

A central support can comprise a notch 44 in the middle of the support, so as to leave the frenulum of the lip present at the user's upper and lower arches free. The notch improves user comfort when the dental retractor is in the service position. The notch further enables the spacer 14 to be moved in the user's mouth, in particular to rotate around the dental arches to acquire photos or 3D models in side views. The notch may, for example, be triangular, rounded or rectangular in shape. The length of the notch is preferably greater than 3 mm, greater than 5 mm, even greater than 10 mm, even greater than 15 mm, and/or less than 50 mm, preferably less than 30 mm, preferably less than 20 mm.

Each central support preferably has a length L₂₄ₛ, measured along the main direction of the central support, greater than 1 cm, preferably greater than 2 cm, preferably greater than 3 cm and/or less than 15 cm, preferably less than 12 cm, preferably less than 8 cm. The length of the upper and lower supports is preferably the same.

Like the central supports, the lateral supports are shaped to be interposed between the lips and the teeth.

Remarkably, the central and/or lateral supports are not shaped like channels. They offer only a limit to the movement of the lips toward the teeth, thus exposing the latter, but allow the lips to move in the opposite direction. Preferably, they offer no obstacle to said movement in said opposite direction, away from the teeth. Comfort is considerably enhanced when users need to acquire 2D or 3D representations from different viewpoints, and need to move the mobile phone to the right, left, up or down.

Also remarkably, the lateral supports have not only a tooth-spacing function, but also a cheek-spacing function. Their long length enables highly effective cheek spacing, as shown in FIG. 4.

The free end 30 of the supports (referenced 30s, 30i, 30d and 30g for upper, lower, right and left supports, respectively) is preferably rounded, so as to be non-traumatic.

Preferably, the supports extend outside a theoretical tube extending along the outer surface of the tubular part and extending beyond the oral opening.

Each support defines the free end of a respective tab, the opposite end of which is connected to the non-deformable portion. The area at the interface between a tab and the deformable portion is called “junction” 32. The junction and/or the shape of the tab are shaped so that each tab can be elastically moved away from its position in the rest configuration, so that its free end is pushed toward the axis X, and so that the area of the oral opening can be reduced. The arrow F in FIG. 6 shows a force to exert a centripetal thrust to move the free end 30g of the left tab toward the axis X.

Preferably, the set of tabs, consisting of a right tab 34d, a left tab 34d, a top tab 34s and a bottom tab 34i, defines the deformable portion.

The upper 34s and lower 34i tabs, that is, the central tabs, can carry stiffening ribs 36.

The gap between two adjacent tabs is called a “slit” 38.

Preferably, the approaches of the two lateral tabs, that is, carrying the lateral supports, and the two center tabs, that is, carrying the center supports, are coupled, that is, an approach of the two lateral tabs toward one another causes the two center tabs to approach one another, or an approach of the two center tabs toward one another causes the two lateral tabs to approach one another.

The coupling member is preferably a pin 39 integral with the flap 40 of a tab, resting on an adjacent tab, and shaped to exert an increasingly strong centripetal thrust as said tab approaches the axis X. The coupling member can rest on a boss 41, or “cam”, of the adjacent tab.

In the embodiment shown, each lateral tab carries two said flaps, resting on the two central tabs, respectively. Pinching to bring the two lateral tabs together therefore leads to a double action on each central tab, by each of the lateral tabs adjacent to said central tab.

This makes it particularly easy to reduce the oral opening, especially when arranging the spacer in the service position.

Preferably, a stop member 42 limits the approach of the two lateral tabs toward one another and the two center tabs toward one another. In particular, the sliding of a flap from a lateral tab onto a central tab can be blocked by contact of the flap on a stop member carried by said central tab.

Advantageously, the stops can be used to define the minimum area of the oral opening, that is, the contracted configuration of the spacer. To reduce the area of the oral opening as much as possible, the user simply pulls the two lateral tabs toward one another and the two central tabs toward one another until they feel the abutment.

The stops also facilitate handling of the spacer, as the user can squeeze the lateral tabs without the risk of breaking the spacer.

In a preferred embodiment, the stop members 42 are attached to the central tabs, preferably in one piece with the central tabs.

Preferably, the central tabs are configured so as, when the respective lateral tabs reach the contracted configuration, to cooperate with said respective lateral tabs so as to retain said lateral tabs, and thus keep them in the contracted position when the user ceases any action bringing the lateral tabs together. The lateral tabs are then in a locked position. For example, each lateral tab may comprise an elastic claw that hooks a respective central tab before reaching the stop position. Preferably, the locking devices, such as the claws, can be unlocked by moving the central tabs toward one another.

FIG. 11 shows one such embodiment. A lateral tab 341 (34d or 34g) is provided with an elastic claw 43 configured to elastically engage in a cavity 45 arranged in a central tab 34c (34s or 34i) when the contracted configuration is reached. This engagement thus prevents the lateral tab from sliding back into the rest configuration (arrow F1), and thus keeps it in the contracted configuration.

When the central tab is brought closer to the axis X (arrow F3), the claw 43 disengages, allowing the lateral tab to return to the rest configuration (unlocked).

Preferably, the free end of the claw 43 is the part of the lateral tab that comes into abutment with the stop member 42 of the central tab, so as to define the contracted configuration and prevent sliding, along arrow F2, of the lateral tab beyond the contracted configuration.

Preferably, each flap is a masking member that extends between two adjacent tabs and is shaped to mask the space between these two tabs, or “slits”, that separate the two tabs. In FIG. 6, in the rest configuration, the flap 40 comes to the limit of the central tab 34s. It is separated only by the slit 38. Once the user squeezes the deformable portion by pressing on the two lateral tabs, the flap 40 covers the slit 38, then slides over the central tab, this sliding contact pressing on the central tab to bend it toward the axis X, as shown in FIG. 7.

The masking of the space extending between two adjacent tabs advantageously limits the entry of light into the chamber of the tubular part, which facilitates the acquisition of good quality images.

In FIG. 6, the cutout in the deformable portion to define the flap of the right tab has been highlighted with a very thick line C₄₀. In other words, the slit delimiting the right tab has the general shape of an “L”, extending partially in a plane passing through the axis X, then continuing in a plane perpendicular to the axis X. Alternatively, the slit may have the general shape of a “T”, the crossbar of which preferably extends in a plane passing through the axis X.

FIG. 8 shows that the junction between said tab and the non-deformable portion, in the plane of the oral opening Oo, thus has a width I₃₂ less, preferably at least 1.1 times less, preferably at least 1.2 times less than the width I_{34g} of the tab at mid-length of the tab, which takes into account the presence of the flaps.

Preferably, the lateral tab rests on the central tab for more than 50%, more than 70%, more than 90%, preferably substantially 100% (corresponding to permanent contact) of the travel of the flap between the rest and contracted configurations.

### Adapter

The adapter 16 detachably attaches the mobile phone to the spacer, and positions it precisely and easily in an acquisition position enabling the objective 18 of the mobile phone 12 to observe the oral opening Oa through the acquisition position Oa. Preferably, the adapter 16 is configured so that this position can be changed.

One or more elastic bands, one or more straps, one or more hook-and-loop fasteners, one or more clips, one or more screws, a door, for example rotatably mounted on a base of the adapter and lockable in a position wherein it clamps the mobile phone 12, jaws, one or more suction cups or one or more magnets can be used to attach the mobile phone 12 to the spacer or to the adapter 16. The mobile phone can also be inserted into a shell, case or box, attached to a base of the spacer or adapter. This list of examples is not exhaustive.

Even more preferably, the adapter 16 is configured to allow the attachment of different mobile phones, and in particular mobile phones with different dimensions.

In a preferred embodiment, the mobile phone 12 is held on the adapter or spacer 16 by clamping. In particular, preferably as shown in FIG. 1, the adapter 16 comprises a base 46, a first jaw 48₁ and a second jaw 48₂, opposite the first jaw, configured to grip and clamp the mobile phone 12, preferably by pressing on the side edge of the mobile phone.

In one embodiment, the adapter 16 is permanently rigidly attached to the spacer 14, or is even integral with the spacer. However, the adapter 16 is preferably detachably rigidly attached to the spacer 14.

Advantageously, the spacer 14 can be cleaned or sterilized independently of the adapter.

Preferably, the spacer comprises one end housed, preferably nested, in a complementary-shaped housing defined by the adapter.

Preferably, the spacer is shaped in such a way that the deformability of the deformable portion is not altered by assembling the spacer to the adapter and/or by attaching the mobile phone to the spacer, either directly on the spacer or via an adapter.

### Operation

The operation of the kit follows directly from the foregoing description.

Initially, the kit is in the disassembled position. The user clips the spacer 14 onto the adapter 16, inserting the end of the spacer 14 opposite the oral opening Oa into the complementary-shaped housing defined by the adapter. The clipping is preferably achieved by hooking adapter claws into recesses arranged on the outer surface of the spacer 14.

After clipping, the spacer and the adapter together define the chamber 26.

The user then clamps the mobile phone 12 between the first and second jaws so that the acquisition opening Oa is substantially opposite the objective 18 and preferably the flash. The kit, in the acquisition position, is then ready for use.

The spacer is then in its rest position. In this configuration, the user cannot fit all the supports in his mouth.

The user then presses the two lateral tabs toward one another, which pushes the two central tabs toward one another, until the flaps of the lateral tabs come into abutment with corresponding stop members of the central tabs. The area of the oral opening is then minimal, and the spacer is in the contracted configuration.

In this configuration, the user can then insert all of the supports between his lips and teeth. After this insertion, the user releases the pressure on the two lateral tabs. The spacer then tends to return elastically to the rest configuration. However, this return is hampered by the action of the cheeks on the lateral supports, and by the action of the central parts of the lips and the corners of the mouth on the central and lateral supports, respectively. These elastic actions effectively spread the cheeks and lips away from the teeth, allowing the mobile phone objective to have an unobstructed view of the teeth.

Additionally, the elasticity allows the spacer to adapt to the user's morphology, so that the spacer can be available in just one size for the entire population.

The user then positions the oral opening in front of the teeth for which he wants to acquire one or more 2D or 3D representations, preferably taking photos or acquiring a scan. He is then in a service position.

By pressing the shutter release of the mobile phone, the user acquires a 2D or 3D representation.

If required, the user then moves the oral opening to acquire 2D or 3D representations in other orientations of the mobile phone relative to the user's teeth. In particular, by rotating the kit around the user, the mobile phone can observe different regions of the user's mouth, for example to take photos of the dental arch or the teeth at the back of the mouth.

To facilitate this movement, the user can advantageously squeeze the lateral tabs toward one another again, to contract the oral opening. Preferably, the contracted configuration is defined by one or more stop members so that, in the contracted configuration, a gap remains between adjacent supports. Preferably, in the stop configuration, that is, the contracted configuration, the distance between any lateral support and each of the central supports adjacent to said lateral support is greater than 1 mm, preferably greater than 2 mm, preferably greater than 5 mm, preferably greater than 8 mm, preferably greater than 10 mm, and/or less than 30 mm, preferably less than 20 mm. Advantageously, the user thus runs no risk of pinching himself, and in particular of pinching a cheek or his tongue, between the central tabs and the lateral tabs.

Preferably, the user acquires 2D or 3D representations by acquiring at least a front view, a right view and a left view.

The spacer does not prevent the user's jaws from moving toward or away from one another. It thus allows the acquisition of photos showing the user's teeth, with the mouth open or closed, while keeping the lips curled up.

Preferably, the user acquires 2D or 3D representations by acquiring at least one open-mouth view and at least one closed-mouth view, preferably for front, right and left views in each case.

When the user turns the spacer to the right (or left), the right (or left, respectively) corner of the mouth slides into sliding zone 50d. This area is free of obstructions, which makes sliding easier and the spacer particularly comfortable. The length of the sliding zone Lsoa advantageously allows a wide range of spacer rotation without the right (or left, respectively) corner of the mouth escaping and obstructing the view of the optical objective.

The user then removes the spacer 14 from his mouth, and disassembles the mobile phone.

Of course, the order of operations can be changed. For example, the user can attach the spacer to the adapter before or after attaching the mobile phone. The spacer can be removed before or after the mobile phone.

As is now clear, the invention limits the risk of acquiring photos or 3D models with a spacer that is unsuitable for the user.

The kit enables fast, hygienic acquisition, typically in under a minute, without the need for a specialist, such as a dentist or orthodontist. In particular, acquisition can be carried out by the user himself or by one of his family members, using a simple mobile phone, anywhere, and in particular outside a medical, dental or orthodontic practice. Additionally, acquisition is possible without the use of a tool that rests on the ground to immobilize the mobile phone, and in particular without a tripod.

Last but not least, the kit enables fast, reliable attachment of the mobile phone, whatever its thickness, without the risk of damaging it. It can be implemented as part of the method disclosed in PCT/EP2015/074896.

Of course, the invention is not limited to the embodiments disclosed and shown, which are provided for illustrative purposes only.

In particular, the shape of the spacer is not limiting. In particular, in one embodiment, the chamber opens only through the oral and acquisition openings. This is not an exhaustive list, however, as the chamber may have other openings.

The masking and coupling elements are not necessarily the same element (flap).

The modification of the oral opening by deformation of the deformable portion is not limited to deformation by tab bending. It could, for example, result from rotation of the tabs around their junctions with the non-deformable portion, or from the use of a mechanical mechanism.

It would also be possible to use a system with metal springs and to insert supports, or to use a system with plastic springs and to insert supports.

### LIST OF REFERENCES

10: kit
12: mobile phone
14: spacer
16: adapter
18: mobile phone optical objective
20: mobile phone screen
22: tubular part
22₁: non-deformable portion
22₂: deformable portion
24: support, generically
24d: right support
24g: left support
24s: upper support
24i: lower support
26: chamber of the tubular part
28: fastener
30: free end of support, generically
30d: free end of right support
30g: free end of left support
30s: free end of upper support
30i: free end of lower support
32: junction, generically
32s: junction of the upper tab
32g: junction of the left tab
34: tab, generically
34d: right tab
34g: left tab
34s: upper tab
34i: lower tab
36: stiffening rib
38: slit
39: (coupling) pin
40: flap, coupling member, masking member
41: boss
42: stop member
44: notch
46: base
48₁: first jaw
48₂: second jaw
50d: sliding zone of the right corner of the mouth
Oo: oral opening
Oa: acquisition opening
X: longitudinal axis of the spacer
F: thrust force
L₂₂: length of tubular part
I₂₂: width of tubular part
h₂₂: height of tubular part
L₂₄ₛ: length of upper support
I₃₂: width of a junction 32
I_{34g}: width of the tab 34g at mid-length of the tab
L_{50d}: length of the sliding zone of the right corner of the mouth
C₄₀: line along the cutout of a flap
Dx₃₀′: distance of a free end of a support (30g), in the direction of the axis X, between the general plane of the oral opening and the free end of said support
Dy₃₀: distance between the axis X and a free end of a support, along the axis Oy), in the rest configuration
Dy₃₀′: distance between the axis X and a free end of a support, along the axis Oy), in the contracted configuration
Dz₃₀: distance between the axis X and a free end of a support, along the axis Oz), in the rest configuration
Dz₃₀′: distance between the axis X and a free end of a support, along the axis Oz), in the contracted configuration

1. A one-piece spacer intended for the acquisition, with a mobile telephone, of 2D or 3D representations of a user's teeth, the spacer comprising a tubular part having a longitudinal axis, said tubular part:
having a length greater than 3 cm;
opening through an acquisition opening and through an oral opening;
being shaped to be fixed, removably, to the mobile telephone, in an acquisition position in which an optical lens of the mobile telephone has an at least partial view of the oral opening through the opening of acquisition, and
comprising a non-deformable section opening through the acquisition opening and, in the extension of the non-deformable section, a deformable section opening through the oral opening and deformable between a rest configuration in which the oral opening has a maximum area and a contracted configuration in which the oral opening has a minimum area, the ratio of the minimum area to the maximum area being less than 0.95.

2. The spacer according to the claim 1, wherein said ratio of the minimum area to the maximum area is less than 0.7.

3. The spacer according to claim 1, in which the deformable section comprises a plurality of tabs, each tab:
partially delimiting the oral opening;
being physically and exclusively connected to the non-deformable section by a junction at one end of said tongue, and
being deformable or elastically foldable under the effect of a thrust oriented towards said longitudinal axis between the rest and contracted configurations.

4. The spacer according to claim 3, comprising a stop member arranged so as to block the movement of the tongues towards the longitudinal axis when said tongues reach the contracted configuration.

5. The spacer according to claim 4, in which the stop member is arranged so, in said contracted configuration, each tongue is spaced apart by a distance greater than 1 mm, preferably greater than 2 mm, preferably greater at 3 mm, preferably greater than 5 mm, preferably greater than 8 mm, preferably greater than 10 mm, and less than 30 mm, preferably less than 20 mm, from each of the adjacent tongues.

6. The spacer according to claim 3, comprising a coupling member of the movement of at least one tongue with the movement of at least one adjacent tongue, from the rest configuration to the compacted configuration, and in particular, in the preferred embodiment so that the movement of the side tabs causes the movement of the central tabs.

7. The spacer according to claim 3, comprising a masking flap configured to completely mask the space extending between two adjacent tongues, in particular between any central tongue and at least one, preferably each lateral tabs adjacent to the central tab, said flap preferably constituting said coupling member, the masking preferably being permanent from the rest configuration to the contracted configuration.

8. The spacer according to claim 3, in which at least one tab, preferably each tab comprises a support shaped to, in a service position, keep part of the lips of the user away from the user's teeth. user, at least one said support is shaped so as to authorize, in the service position, sliding of the user's lips away from the user's teeth, preferably over a distance greater than 3 mm, preferably greater than 5 mm, preferably greater than 10 mm, and/or less than 100 mm.

9. The spacer according to claim 3, in which the tubular part is made of a transparent material.

10. The spacer according to claim 3, in which the deformable section comprises, preferably consists of
said right and left side tabs comprising right and left side supports shaped to, in a service position, move the right and left corners away from the user's teeth of the user's lips, respectively; and
said upper and lower central tabs comprising upper and lower supports shaped to, in the service position, move the upper and lower central parts away from the user's teeth of the user's lips, respectively.

11. The spacer according to claim 10, in which
a central tongue comprises said stop member, said stop member being arranged in such a way that a lateral tongue, preferably a masking flap integrated in a side tongue, comes into abutment with said stop member when said side tongue reaches the contracted configuration, and/or
a lateral tab comprises said coupling member, preferably a masking flap, said coupling member being supported on at least one central tab, preferably for more than 50%, more than 70%, more than 90%, preferably substantially 100% (that is to say in permanent support) of the travel of said lateral tongue between the rest and contracted configurations.

12. The spacer according to claim 10, wherein at least one, preferably each side support has a free end which, in the rest configuration, extends to a distance from the longitudinal axis greater than 45 mm so as to elastically spread one cheek of the user in the service position.

13. The spacer according to claim 10, in which each lateral support deviates progressively from the longitudinal axis from the oral opening to a free end of said lateral support.

14. The spacer according to claim 1, comprising a mechanism for maintaining the deformable section in the contracted configuration, preferably activatable and deactivatable by moving the tabs when the spacer has the deformable section comprising a plurality of tabs, each tab:
partially delimiting the oral opening;
being physically and exclusively connected to the non-deformable section by a junction at one end of said tongue; and
being deformable or elastically foldable under the effect of a thrust oriented towards said longitudinal axis between the rest and contracted configurations.

15. A kit for acquiring 2D or 3D representations of a user's teeth, comprising:
a mobile telephone provided with an optical objective for acquiring a said 2D representation and/or a said 3D representation; and
a spacer according to claim 1, on which the mobile phone is fixed, preferably in a removable manner, optionally via an adapter, the mobile phone being fixed on the spacer so that the optical lens has at least a partial view of the oral opening through the acquisition opening.

16. Method for acquiring a 2D or 3D representation by means of an acquisition kit according to claim 15, said method comprising the following steps: a) partial introduction, by the user, of the spacer into the user's mouth to a service position in which the optical lens of the mobile phone has a view of the user's teeth through the oral opening, the supports of the spacer spreading the lips of the user to release said teeth; b) activation, preferably by the user, of the mobile phone so as to acquire said 2D or 3D representation; c) modification of the positioning of the oral opening relative to the user, by rotation around the user, then resumed in step b).
